(19) United States
(12) Patent Application Publication
(10) Pub. No.: US 2017/0112738 A1
(43) Pub. Date: Apr. 27, 2017

(54) COSMETIC INCLUDING ISOPARAFFIN

(71) Applicant: Henkel AG & Co. KGaA, Duesseldorf (DE)

(72) Inventors: Jens Delowsky, Norderstedt (DE); Thomas Hippe, Appen (DE); Edith von Aspern, Hanstedt (DE); Bernd Richters, Hamburg (DE)

(73) Assignee: Henkel AG & Co. KGaA, Duesseldorf (DE)

(21) Appl. No.: 15/399,587

(22) Filed: Jan. 5, 2017

Related U.S. Application Data
(63) Continuation of application No. PCT/EP2015/064116, filed on Jun. 23, 2015.

(30) Foreign Application Priority Data
Jul. 7, 2014 (DE) 10 2014 213 136.7
Nov. 28, 2014 (DE) 10 2014 224 453.6

(51) Int. Cl. A61K 8/37; A61Q 5/00; A61K 8/31

(52) U.S. Cl. CPC A61K 8/37; A61K 8/31; A61Q 5/00; A61K 2800/34

(57) ABSTRACT

The invention relates to cosmetics which include isoparaffin, are preferably silicone-free and are preferably in the form of a hair oil. Also disclosed is the use of said cosmetics for keratin fiber care.

### FIELD OF THE INVENTION

The present invention generally relates to cosmetics which include isoparaffin, are preferably silicone-free and are preferably in the form of a hair oil, and to the use of said cosmetics for keratin fiber care.

### BACKGROUND OF THE INVENTION

Besides natural environmental influences, human hair is exposed to a number of other stresses, particularly cosmetic stresses. These stresses which damage the hair include for example coloring the hair and also styling the hair, for example by way of a perm. Cosmetic hair care agents are used to alleviate the disadvantageous effects of the (environmental) influences that adversely affect the hair structure, but also to maintain and improve the natural hair structure. Organosilicon compounds, particularly silicones such as trisiloxanes which are characterized by caring properties, are an important active substance in many of these cosmetics. The disadvantages of these silicones are the reduced penetration of active substances and auxiliaries into the hair as a result of the wetting of the hair surface, and also the fact that hairstyles are more difficult to create due to the wetting of the hair surface. Providing low-silicone or silicone-free care agents is therefore a relevant aim in the field of hair cosmetics.

In the prior art, isoparaffins are discussed as one of many active substance classes suitable as a substitute for silicones. For example, European patents EP 413417 B1 (Colgate Palmolive) and EP 1284712 B1 (Procter & Gamble) describe hair shampoos and hair conditioners with a caring effect which, besides further constituents, may also include isoparaffins. However, these hair care agents still have a number of disadvantages and, despite what has been achieved thus far, there is still a need for low-trisiloxane or trisiloxane-free hair care agents based on isoparaffin.

The use of ester oils in hair conditioning agents is described in international patent application WO 2010/063565 A1 (Henkel).

EP 2623088 A2 (Henkel) discloses hair care agents which include at least one ester oil and at least one isoparaffin from the group consisting of isodecane, isoundecane, isododecane, isotridecane and isotetradecane. Mixtures of ester oils and polymeric quaternary ammonium compounds are also described, as well as combinations of both cosmetics.

The object of the present invention is to provide cosmetics which render superfluous the co-utilization of organosilicon compounds, particularly silicones, and can omit the use thereof and nevertheless retain the performance spectrum of the silicone-including formulations. The intention is to avoid the weightiness and greasiness that often occurs on the treated hair when silicones are used.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic including, in a cosmetically acceptable carrier, at least one isoparaffin from the group consisting of isodecane, isoundecane, isododecane, isotridecane and isotetradecane; at least one ester oil from the group consisting of tri-fatty acid esters of saturated and/or unsaturated linear and/or branched fatty acids with glycerol; and at least one ester oil c) different from ester oil b).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The object is achieved according to the invention by a cosmetic including, in a cosmetically acceptable carrier,
a) at least one isoparaffin from the group consisting of isodecane, isoundecane, isododecane, isotridecane and isotetradecane,
b) at least one ester oil from the group consisting of tri-fatty acid esters of saturated and/or unsaturated linear and/or branched fatty acids with glycerol,
c) at least one ester oil c) different from ester oil b).

It has been found according to the invention that, by combining at least one isoparaffin a) with at least one ester oil b) and at least one further ester oil c), it is possible to obtain cosmetics which do not lead to a weightiness or greasiness of the hair treated therewith and which moreover ensure good combability and shine of the hair. It is thus possible to obtain cosmetics, particularly hair care agents, which exhibit an excellent care effect and in particular give rise to good shine properties, an improved appearance and preferably improved regeneration of the keratin fibers.

As the component a), the cosmetics include at least one isoparaffin from the group consisting of isodecane, isoundecane, isododecane, isotridecane and isotetradecane. Use may be made of individual isoparaffins or of mixtures of two or more of the aforementioned isoparaffins. Compared to the n-paraffins having the same number of carbon atoms, these isoparaffins are characterized by improved olfactory properties when used in the cosmetics. Compared to the n-paraffins having the same number of carbon atoms and also compared to the isoparaffins having a lower or higher number of carbon atoms, an improved cosmetic effect, in particular hair care effect, is moreover surprisingly achieved in combination with the ester oils.

The use of isoparaffins from the group consisting of isoundecane, isododecane and isotridecane has proven to be particularly advantageous for the cosmetic effect. Such cosmetics, characterized in that they include at least one isoparaffin selected from the group consisting of isoundecane, isododecane, isotridecane, are preferred according to the invention.

Cosmetics according to the invention which include a mixture of isoundecane, isododecane and isotridecane as the isoparaffin exhibit particularly advantageous effects in combination with the oil bodies. In one preferred embodiment, cosmetics according to the invention are therefore characterized in that they include as the isoparaffin a mixture of isoundecane, isododecane and isotridecane, in particular a mixture of isododecane and isotridecane.

The proportion by weight of the isoundecane, isododecane and isotridecane based on the total weight of all the isoparaffins used in the cosmetic is preferably more than 62% by weight, more preferably more than 72% by weight, particularly preferably more than 82% by weight and in particular more than 92% by weight. It is particularly preferred that the proportion by weight of the isododecane and isotridecane based on the total weight of all the isoparaffins used in the cosmetic is more than 60% by weight, preferably more than 70% by weight, particularly preferably more than 80% by weight and in particular more than 90% by weight.

The density at 15° C. (DIN 51 757 P.4; ASTM D 4052) of preferred isoparaffin mixtures is preferably 700 to 820 kg/m³, more preferably 720 to 800 kg/m³ and in particular 740 to 780 kg/m³.

Preferred isoparaffin mixtures are characterized by a boiling range (DIN ES ISO 3405; ASTM D 86) between 195° C. and 250° C., preferably between 200° C. and 245° C. and in particular between 205° and 240° C. One isoparaffin mixture which is particularly preferred according to the invention is available commercially under the name Pionier® 2094 (H&R Group). The at least one isoparaffin a) is particularly preferably isododecane.

The at least one ester oil b) is selected from the group consisting of tri-fatty acid esters of saturated and/or unsaturated linear and/or branched fatty acids with glycerol. Preferably, the at least one ester oil b) is selected from tri-fatty acid esters of saturated linear and/or branched C₆₋₁₂ carboxylic acids with glycerol. The use of linear C₆₋₁₂ carboxylic acids, in particular C₈₋₁₀ carboxylic acids, is particularly preferred. With particular preference, the at least one ester oil b) is a mixture of tri-fatty acid esters of caprylic acid and capric acid with glycerol, preferably in a weight ratio of the caprylic acid triglyceride to the capric acid triglyceride of 3:1 to 1:1.

The at least one ester oil c) is different from the at least one ester oil b). The ester oil c) thus cannot be an ester oil that is already present in component b).

The ester oils c) are defined as follows: Ester oils c) will be understood to mean the esters of C₆-C₃₀ fatty acids with C₂-C₃₀ fatty alcohols. Preference is given to the monoesters of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and technical mixtures thereof. Examples of fatty alcohol components in the ester oils are isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol, and technical mixtures thereof.

According to the invention, particular preference is given to isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

Of course, the ester oils may also be alkoxylated with ethylene oxide, propylene oxide or mixtures of ethylene oxide and propylene oxide. The alkoxylation may in this case be found either on the fatty alcohol part or on the fatty acid part or on both parts of the ester oils. However, it is preferred according to the invention if the fatty alcohol has first been alkoxylated and then esterified with fatty acid. These compounds are shown in general in formula (D4-I).

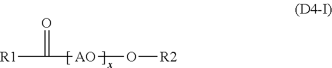

Here, R1 represents a saturated or unsaturated, branched or unbranched, cyclic saturated cyclic unsaturated acyl radical having 6 to 30 carbon atoms,
AO represents ethylene oxide, propylene oxide or butylene oxide,
X represents a number between 1 and 200, preferably 1 and 100, particularly preferably between 1 and 50, very particularly preferably between 1 and 20, extremely preferably between 1 and 10 and most preferably 1 and 5,
R2 represents a saturated or unsaturated, branched or unbranched, cyclic saturated cyclic unsaturated alkyl, alkenyl, alkynyl, phenyl or benzyl radical having 6 to 30 carbon atoms. Examples of fatty acid components used as radical R1 in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and technical mixtures thereof. Examples of fatty alcohol components as radical R2 in the ester oils are benzyl alcohol, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol, and technical mixtures thereof. One ester oil which is particularly preferred according to the invention is available for example under the INCI name PPG-3 Benzyl Ether Myristate, for example the commercial product Crodamol® STS.

The following are also to be understood as ester oils:
dicarboxylic acid esters such as di-n-butyl adipate, di-(2-ethylhexyl) adipate, di-(2-ethylhexyl) succinate and diisotridecyl acelaate and also diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate, and also
symmetrical, unsymmetrical or cyclic esters of carbonic acid with fatty alcohols, for example glycerol carbonate or dicaprylyl carbonate (Cetiol® CC),
fatty acid partial glycerides, that is to say monoglycerides, diglycerides and technical mixtures thereof. If technical products are used, only small amounts of triglycerides can be included therein for manufacturing reasons. The partial glycerides preferably follow the formula (D4-II)

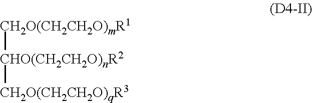

in which R¹, R² and R³ independently of one another represent hydrogen or a linear or branched, saturated and/or unsaturated acyl radical having 6 to 22, preferably 12 to 18, carbon atoms, with the proviso that at least one of these groups represents an acyl radical and at least one of these groups represents hydrogen. The sum (m+n+q) represents 0 or numbers from 1 to 100, preferably 0 or 5 to 25. Preferably, Rⁱ represents an acyl radical and R² and R³ represent hydrogen and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and technical mixtures thereof. Oleic acid monoglycerides are used with preference.

Preferably, however, the at least one ester oil c) is selected from symmetrical, unsymmetrical or cyclic esters of carbonic acid with fatty alcohols. With particular preference, the at least one ester oil c) is selected from esters of carbonic acid with C₆₋₁₂ alkanols, in particular C₇₋₉ alkanols. These are particularly preferably symmetrical esters of carbonic acid with alkanols, especially dicaprylyl carbonate (ester of carbonic acid with n-octyl alcohol).

As component c), use may also be made of mixtures of two or more of the aforementioned ester oils of component c). Preference is given to the presence of at least one symmetrical, unsymmetrical or cyclic ester of carboxylic acid with fatty alcohols.

With particular preference, in mixtures of ester oils c), there is a majority amount of symmetrical, unsymmetrical or cyclic esters of carbonic acid with alcohols. According to one embodiment of the invention, the at least one ester oil c) includes only symmetrical, unsymmetrical or cyclic esters of carbonic acid with fatty alcohols.

The cosmetics according to the invention may include the components a), b) and c) in any suitable amounts.

Preferred cosmetics include
9.8 to 99.6% by weight, preferably 40 to 98.5% by weight, particularly preferably 70 to 98.5% by weight, in particular 85 to 94% by weight component a),
0.2 to 90% by weight, preferably 0.5 to 40% by weight, particularly preferably 0.5 to 10% by weight, in particular 1 to 5% by weight component b) and
0.2 to 90% by weight, preferably 1 to 40% by weight, particularly preferably 1 to 20% by weight, in particular 5 to 10% by weight component c).

Based on the cosmetic, the total amount of which amounts to 100% by weight, the sum of components a), b) and c) preferably accounts for 10 to 99.9% by weight, preferably 50 to 99% by weight, in particular 90 to 99.8% by weight.

Preferred cosmetics include, based on their total weight, components a), b) and c) in weight proportions above 90% by weight, preferably above 94% by weight and in particular above 98% by weight. In one preferred embodiment, the cosmetics consist entirely of components a), b) and c).

By choosing suitable amounts of components b) and c), the cosmetic according to the invention can be tailored to the respective hair quality. Formulations for treating fine, normal or thick hair can thus be obtained in a targeted manner.

By using the cosmetics according to the invention, it is possible to omit organosilicon compounds from the cosmetics, particularly silicones such as trisiloxanes. Preferred embodiments according to the invention are therefore characterized in that
the cosmetic, based on its total weight, includes less than 5.0% by weight, preferably less than 2.0% by weight, more preferably less than 0.5% by weight and in particular no trisiloxane; and/or
the cosmetic, based on its total weight, includes less than 5.0% by weight, preferably less than 2.0% by weight, more preferably less than 0.5% by weight and in particular no dimethicone; and/or
the cosmetic, based on its total weight, includes less than 5.0% by weight, preferably less than 2.0% by weight, more preferably less than 0.5% by weight and in particular no amodimethicone; and/or
the cosmetic, based on its total weight, includes less than 5.0% by weight, preferably less than 2.0% by weight, more preferably less than 0.5% by weight and in particular no cyclomethicone; and/or
the cosmetic, based on its total weight, includes less than 5.0% by weight, preferably less than 2.0% by weight, more preferably less than 0.5% by weight and in particular no organosilicon compounds.

Preferably, the cosmetic is free of organosilicon compounds, in particular free of silicones, trisiloxanes and silicone oils.

The composition of some preferred cosmetics can be found in the following tables (unless indicated otherwise, figures specified in % by weight relate to the total weight of the cosmetic).

| | Formula 1 | Formula 2 | Formula 3 | Formula 4 | Formula 5 |
|---|---|---|---|---|---|
| Isoparaffin¹⁾ | 9.8 to 99.6 | 40 to 98.5 | 70 to 98.5 | 85 to 94 | 85 to 94 |
| Ester oil b) | 0.2 to 90 | 0.5 to 40 | 0.5 to 10 | 1.0 to 10 | 1.0 to 5.0 |
| Ester oil c) | 0.2 to 90 | 1 to 20 | 1 to 20 | 5.0 to 10 | 5.0 to 10 |
| Misc. | add 100 | add 100 | add 100 | add 100 | add 100 |

| | | | | | Formula |
|---|---|---|---|---|---|
| | Formula 6 | Formula 7 | Formula 8 | Formula 9 | 10 |
| Isododecane | 9.8 to 99.6 | 40 to 98.5 | 70 to 98.5 | 85 to 94 | 85 to 94 |
| Ester oil b) | 0.2 to 90 | 0.5 to 40 | 0.5 to 10 | 1.0 to 10 | 1.0 to 5.0 |
| Ester oil c) | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 11 | 12 | 13 | 14 | 15 |
| Isoparaffin¹⁾ | 9.8 to 99.6 | 40 to 98.5 | 70 to 98.5 | 85 to 94 | 85 to 94 |
| Ester oil b)²⁾ | 0.2 to 90 | 0.5 to 40 | 0.5 to 10 | 1.0 to 10 | 1.0 to 5.0 |
| Ester oil c) | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 16 | 17 | 18 | 19 | 20 |
| Isoparaffin* | 9.8 to 99.6 | 40 to 98.5 | 70 to 98.5 | 85 to 94 | 85 to 94 |
| Ester oil b) | 0.2 to 90 | 0.5 to 40 | 0.5 to 10 | 1.0 to 10 | 1.0 to 5.0 |
| Ester oil c)³⁾ | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 21 | 22 | 23 | 24 | 25 |
| Isododecane | 9.8 to 99.6 | 40 to 98.5 | 70 to 98.5 | 85 to 94 | 85 to 94 |
| Ester oil b)²⁾ | 0.2 to 90 | 0.5 to 40 | 0.5 to 10 | 1.0 to 10 | 1.0 to 5.0 |
| Ester oil c) | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 26 | 27 | 28 | 29 | 30 |
| Isododecane | 9.8 to 99.6 | 40 to 98.5 | 70 to 98.5 | 85 to 94 | 85 to 94 |
| Ester oil b) | 0.2 to 90 | 0.5 to 40 | 0.5 to 10 | 1.0 to 10 | 1.0 to 5.0 |
| Ester oil c)³⁾ | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 31 | 32 | 33 | 34 | 35 |
| Isoparaffin * | 9.8 to 99.6 | 40 to 98.5 | 70 to 98.5 | 85 to 94 | 85 to 94 |
| Ester oil b)²⁾ | 0.2 to 90 | 0.5 to 40 | 0.5 to 10 | 1.0 to 10 | 1.0 to 5.0 |
| Ester oil c)³⁾ | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 36 | 37 | 38 | 39 | 40 |
| Isododecane | 9.8 to 99.6 | 40 to 98.5 | 70 to 98.5 | 85 to 94 | 85 to 94 |
| Ester oil b)²⁾ | 0.2 to 90 | 0.5 to 40 | 0.5 to 10 | 1.0 to 10 | 1.0 to 5.0 |
| Ester oil c)³⁾ | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 41 | 42 | 43 | 44 | 45 |
| Isoparaffin * | 9.8 to 99.6 | 40 to 98.5 | 70 to 98.5 | 85 to 94 | 85 to 94 |
| Ester oil b)²⁾ | 0.2 to 90 | 0.5 to 40 | 0.5 to 10 | 1.0 to 10 | 1.0 to 5.0 |
| Dicaprylyl carbonate | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 46 | 47 | 48 | 49 | 50 |
| Isododecane | 9.8 to 99.6 | 40 to 98.5 | 70 to 98.5 | 85 to 94 | 85 to 94 |
| Ester oil b)²⁾ | 0.2 to 90 | 0.5 to 40 | 0.5 to 10 | 1.0 to 10 | 1.0 to 5.0 |
| Dicaprylyl carbonate | 0.2 to 90 | 1 to 20 | 1 to 20 | 5.0 to 10 | 5.0 to 10 |
| Misc. | add 100 | add 100 | add 100 | add 100 | add 100 |

| | Formula | Formula | Formula | Formula | Formula |
|---|---|---|---|---|---|
| | 51 | 52 | 53 | 54 | 55 |
| Isoparaffin¹⁾ | 9.8 to 99.5 | 40 to 98.4 | 70 to 98.3 | 85 to 93.8 | 85 to 93.6 |
| Ester oil b) | 0.2 to 89.9 | 0.5 to 40 | 0.5 to 10 | 1.0 to 9.8 | 1.0 to 5.0 |
| Ester oil c) | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 56 | 57 | 58 | 59 | 60 |
| Isododecane | 9.8 to 99.5 | 40 to 98.4 | 70 to 98.3 | 85 to 93.8 | 85 to 93.6 |
| Ester oil b) | 0.2 to 89.9 | 0.5 to 40 | 0.5 to 10 | 1.0 to 9.8 | 1.0 to 5.0 |
| Ester oil c) | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 61 | 62 | 63 | 64 | 65 |
| Isoparaffin¹⁾ | 9.8 to 99.5 | 40 to 98.4 | 70 to 98.3 | 85 to 93.8 | 85 to 93.6 |
| Ester oil b)²⁾ | 0.2 to 89.9 | 0.5 to 40 | 0.5 to 10 | 1.0 to 9.8 | 1.0 to 5.0 |
| Ester oil c) | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 66 | 67 | 68 | 69 | 70 |
| Isoparaffin * | 9.8 to 99.5 | 40 to 98.4 | 70 to 98.3 | 85 to 93.8 | 85 to 93.6 |
| Ester oil b) | 0.2 to 89.9 | 0.5 to 40 | 0.5 to 10 | 1.0 to 9.8 | 1.0 to 5.0 |
| Ester oil c)³⁾ | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 71 | 72 | 73 | 74 | 75 |
| Isododecane | 9.8 to 99.5 | 40 to 98.4 | 70 to 98.3 | 85 to 93.8 | 85 to 93.6 |
| Ester oil b)²⁾ | 0.2 to 89.9 | 0.5 to 40 | 0.5 to 10 | 1.0 to 9.8 | 1.0 to 5.0 |
| Ester oil c) | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 76 | 77 | 78 | 79 | 80 |
| Isododecane | 9.8 to 99.5 | 40 to 98.4 | 70 to 98.3 | 85 to 93.8 | 85 to 93.6 |
| Ester oil b) | 0.2 to 89.9 | 0.5 to 40 | 0.5 to 10 | 1.0 to 9.8 | 1.0 to 5.0 |
| Ester oil c)³⁾ | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 81 | 82 | 83 | 84 | 85 |
| Isoparaffin * | 9.8 to 99.5 | 40 to 98.4 | 70 to 98.3 | 85 to 93.8 | 85 to 93.6 |
| Ester oil b)²⁾ | 0.2 to 89.9 | 0.5 to 40 | 0.5 to 10 | 1.0 to 9.8 | 1.0 to 5.0 |
| Ester oil c)³⁾ | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 86 | 87 | 88 | 89 | 90 |
| Isododecane | 9.8 to 99.5 | 40 to 98.4 | 70 to 98.3 | 85 to 93.8 | 85 to 93.6 |
| Ester oil b)²⁾ | 0.2 to 89.9 | 0.5 to 40 | 0.5 to 10 | 1.0 to 9.8 | 1.0 to 5.0 |
| Ester oil c)³⁾ | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 91 | 92 | 93 | 94 | 95 |
| Isoparaffin * | 9.8 to 99.5 | 40 to 98.4 | 70 to 98.3 | 85 to 93.8 | 85 to 93.6 |
| Ester oil b)²⁾ | 0.2 to 89.9 | 0.5 to 40 | 0.5 to 10 | 1.0 to 9.8 | 1.0 to 5.0 |
| Dicaprylyl carbonate | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

| | Formula | Formula | Formula | Formula | Formula |
|---|---|---|---|---|---|
| | 96 | 97 | 98 | 99 | 100 |
| Isododecane | 9.8 to 99.5 | 40 to 98.4 | 70 to 98.3 | 85 to 93.8 | 85 to 93.6 |
| Ester oil b)²⁾ | 0.2 to 89.9 | 0.5 to 40 | 0.5 to 10 | 1.0 to 9.8 | 1.0 to 5.0 |
| Dicaprylyl carbonate | 0.2 to 89.9 | 1 to 20 | 1 to 20 | 5.0 to 10 | 5.0 to 10 |
| Plant oil | 0.1 to 10 | 0.1 to 10 | 0.2 to 8.0 | 0.2 to 8.0 | 0.4 to 5.0 |
| Misc. | add 100 | add 100 | add 100 | add 100 | add 100 |

1) at least one isoparaffin from the group consisting of isodecane, isoundecane, isododecane, isotridecane and isotetradecane
2) tri-fatty acid esters of caprylic acid and capric acid with glycerol
3) esters of carbonic acid with C₆₋₁₂ alkanols

The cosmetic according to the invention can be used in any suitable fields of application. Said cosmetics are preferably hair treatment agents, in particular hair oils. The cosmetic is thus particularly preferably in the form of a hair oil.

In this case, the hair oil is particularly preferably applied as a spray oil and is sprayed onto the hair. It may be a pump spray, or else spraying may take place with the aid of propellants. Suitable propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, namely either individually or in combination. Hydrophilic propellant gases, such as for example carbon dioxide, can also advantageously be used within the context of the present invention if the proportion of hydrophilic gases is selected to be low and lipophilic propellant gas (for example propane/butane) is present in excess. Particular preference is given to propane, n-butane, isobutane and mixtures of these propellant gases. It has been found that the use of n-butane as the sole propellant gas may be particularly preferred according to the invention.

Suitable pressurized gas containers are vessels made of metal (aluminum, tinplate, tin), protected or shatterproof plastic, or glass coated with plastic on the outside, wherein the compressive and fracture strength, corrosion resistance, ability to be filled easily and also esthetic aspects, ease of handling, printability, etc. play a role when making a choice.

The invention thus also relates to the use of the cosmetic for keratin fiber care.

As mentioned, the cosmetics according to the invention are particularly suitable for hair care. Another subject matter of the present application is therefore a method for treating keratin fibers, characterized in that a cosmetic according to the invention is applied to dry and/or damp keratin fibers.

The hair treatment agent according to the invention is preferably used immediately before, during or after an oxidative or surfactant-based hair treatment. Within the context of the invention, immediately before the oxidative or surfactant-based hair treatment will be understood to mean an application which is directly followed by the oxidative or surfactant-based hair treatment, wherein the hair treatment agent according to the invention has been rinsed from the hair beforehand or has preferably been left on the hair and the hair is preferably still wet.

Within the context of the invention, “after the oxidative or surfactant-based hair treatment” will be understood to mean an application which either directly follows the oxidative or surfactant-based hair treatment, wherein the hair treatment agent according to the invention is applied to the hair, which is preferably still wet or damp, after the agent having an oxidative or surfactant effect has been rinsed off, or is applied to dry or wet hair only after several hours or days. In both cases, the hair treatment agent according to the invention may be rinsed off again after a leave-in time of a few seconds to 45 minutes or may fully remain on the hair.

The effect of the hair treatment agent according to the invention unfolds even during the oxidative or surfactant-based hair treatment and surprisingly lasts even after the hair treatment agent according to the invention has been intensively washed out.

Another subject matter of the present application is the use of a cosmetic according to the invention for keratin fiber care. The following is claimed in particular: the use of a cosmetic according to the invention
to improve the wet and dry combability of keratin fibers,
to improve the shine of keratin fibers,
to improve the moisture balance of keratin fibers,
to protect the keratin fibers against oxidative damage, particularly in the course of oxidative hair treatments,
to prevent keratin fibers from becoming greasy,
to increase the wash resistance of colored keratin fibers,
to achieve increased hairstyle hold for at least 48 h, in particular 24 h,
to improve the so-called curl retention of keratin fibers,
to improve the feel of keratin fibers,
to improve the regeneration of keratin fibers.

According to the invention, combability will be understood to mean both the combability of the wet fibers and also the combability of the dry fibers. The applied combing work or the applied force during the operation of combing a fiber collective serves as a measure of the combability. The measurement parameters can be assessed in a sensory manner by a person skilled in the art or can be quantified by measuring devices.

According to the invention, an oxidative hair treatment defines the effect of an oxidative cosmetic, including at least one oxidizing agent in a cosmetic carrier, on hair.

Preferred cosmetics according to the invention further include specific siloxanes in addition to the ingredients a), b) and c) described above. One preferred subject matter of this application is therefore a cosmetic including, in a cosmetically acceptable carrier,
a) at least one isoparaffin from the group consisting of isodecane, isoundecane, isododecane, isotridecane and isotetradecane,
b) at least one ester oil from the group consisting of tri-fatty acid esters of saturated and/or unsaturated linear and/or branched fatty acids with glycerol,
c) at least one ester oil c) different from ester oil b),
d) at least one trimethylsiloxysilicate and
e) at least one polyalkylsilsesquioxane.

Besides the amount limits for ingredient a) mentioned above, the cosmetics including constituents d) and e) are preferably characterized in that
the proportion by weight of the isoundecane, isododecane and isotridecane, in particular of the isododecane, based on the total weight of the cosmetic is 60 to 99% by weight, preferably 65 to 95% by weight and in particular 70 to 90% by weight;
the proportion by weight of the ester oil b) based on the total weight of the cosmetic is 0.1 to 20% by weight, preferably 0.5 to 15% by weight and in particular 1.0 to 10% by weight;
Preferred cosmetics are characterized in that the proportion by weight of the ester oil c) based on the total weight of the cosmetic is 0.1 to 20% by weight, preferably 0.5 to 15% by weight and in particular 1.0 to 10% by weight.

As component d), the cosmetics which are preferred according to the invention include at least one trimethylsiloxysilicate. Here, the term “trimethylsiloxysilicate” denotes co-hydrolysis products of tetraalkoxysilane and trimethylethoxysilane. These products form a three-dimensional network of polysilicic acid units which are terminated by trimethylsilyl groups, wherein in some circumstances it includes a small amount of ethoxy and hydroxyl functions. The average molecular weight of the trimethylethoxysilane can be determined from the ratio of the tetraalkoxysilane units to the trimethylethoxysilane units. Preferably, this ratio is 0.5 to 1.0, particularly preferably 0.66. One example of a trimethylethoxysilane with a ratio of 0.66 is Wacker-Belsil TMS 803 from Wacker-Chemie GmbH. One exemplary formula for trimethylsiloxysilicate is

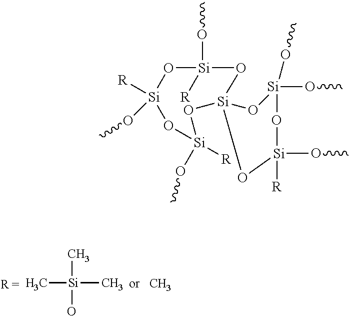

Trimethylsiloxysilicate is a water-insoluble additive which can be used as a film-forming agent and fixative. Cosmetics which are preferred according to the invention include trimethylsiloxysilicate within narrow amount ranges. Preferred cosmetics according to the invention are characterized in that they include, based on their total weight, 0.3 to 7.0% by weight, preferably 0.4 to 5.0% by weight and in particular 0.6 to 3.5% by weight trimethylsiloxysilicate.

As component e), at least one polyalkylsilsesquioxane is used in the cosmetics which are preferred according to the invention. The term “polyalkylsilsesquioxanes” refers here to compounds from the group of polyhedral oligomeric silsesquioxanes (POSS), which are described by the empirical formula RSiO_{1.5}, in which R is an organic substituent. This organic radical may be for example hydrogen, siloxy or a cyclic or linear aliphatic or aromatic group which may additionally include reactive functional groups such as alcohol, ester, amine, keto, olefin, ether or halide groups. The basic structure of POSS compounds has a polyhedral Si—O backbone, to which the R groups are bound. Homoleptic POSS compounds including just one single type of R groups and heteroleptic POSS chemicals including respectively different R groups are known.

Polyalkylsilsesquioxanes are used according to the invention, that is to say the radicals R are alkyl radicals. With particular preference, homoleptic polyalkylsilsesquioxanes are used according to the invention, that is to say just one type of alkyl radical is included in the molecule.

By way of example, the following polyalkylsilsesquioxanes can be used according to the invention: isooctyl-POSS [Me₃CCH₂CH(Me)CH₂]ₙTₙ, where n=8, 10 or 12, octacyclohexyl-POSS C₄₈H₇₂O₁₂Si₈, octacyclopentyl-POSS C₄₀H₇₂O₁₂Si₈, octaisobutyl-POSS C₃₂H₇₂O₁₂Si₈, octamethyl-POSS C₈H₂₄O₁₂Si₈. Very particular preference is given to the use of polypropylsilsesquioxane, that is to say the radical R in the formula RSiO_{1.5} is a propyl radical, wherein “propyl radical” is understood to mean both n-propyl and isopropyl radicals.

Preferred cosmetics according to the invention are characterized in that they include, based on their total weight, 0.2 to 3.0% by weight, preferably 0.3 to 2.0% by weight and in particular 0.4 to 1.5% by weight polyalkylsilsesquioxane.

For the cosmetic properties of the cosmetics according to the invention, it has proven to be advantageous if the weight ratio of components d) and e) is 4:1 to 1:2, preferably 3:1 to 1:1.

By choosing suitable amounts of components a), b), c), d) and e), the cosmetic according to the invention can be tailored to the respective hair quality. Formulations for treating fine, normal or thick hair can thus be obtained in a targeted manner.

One preferred subject matter of the present application is a cosmetic including, in a cosmetically acceptable carrier, in each case based on the total weight of the cosmetic,
a) 65 to 95% by weight of at least one isoparaffin from the group consisting of isodecane, isoundecane, isododecane, isotridecane and isotetradecane,
b) 1.0 to 10% by weight of at least one ester oil from the group consisting of tri-fatty acid esters of saturated and/or unsaturated linear and/or branched fatty acids with glycerol,
c) 1.0 to 10% by weight of at least one ester oil c) different from ester oil b),
d) 0.6 to 3.5% by weight of at least one trimethylsiloxysilicate and
e) 0.4 to 1.5% by weight of at least one polyalkylsilsesquioxane.

Preferred cosmetics include, based on their total weight, the components a), b), c), d) and e) in weight proportions above 90% by weight, preferably above 94% by weight and in particular above 98% by weight. In one preferred embodiment, the cosmetics consist entirely of components a), b), c), d) and e).

The composition of some further preferred cosmetics can be found in the following tables (unless indicated otherwise, figures specified in % by weight relate to the total weight of the cosmetic).

| | Formula | Formula | Formula | Formula | Formula |
|---|---|---|---|---|---|
| | 1a | 2a | 3a | 4a | 5a |
| Iso- | 60 to 99 | 60 to 98 | 65 to 95 | 65 to 95 | 70 to 90 |
| paraffin 1) | | | | | |
| Ester oil b) | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethyl- | 0.3 to 7.0 | 0.3 to 7.0 | 0.4 to 5.0 | 0.4 to 5.0 | 0.6 to 3.5 |
| siloxysilicate | | | | | |
| Polyalkyl- | 0.2 to 3.0 | 0.2 to 3.0 | 0.3 to 2.0 | 0.3 to 2.0 | 0.4 to 1.5 |
| silsesqui- | | | | | |
| oxane | | | | | |
| Misc. | add 100 | add 100 | add 100 | add 100 | add 100 |

| | Formula | Formula | Formula | Formula | Formula |
|---|---|---|---|---|---|
| | 6 | 7a | 8a | 9a | 10a |
| Isododecane | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethyl- | 0.3 to 7.0 | 0.3 to 7.0 | 0.4 to 5.0 | 0.4 to 5.0 | 0.6 to 3.5 |
| siloxysilicate | | | | | |
| Polyalkyl- | 0.2 to 3.0 | 0.2 to 3.0 | 0.3 to 2.0 | 0.3 to 2.0 | 0.4 to 1.5 |
| silsesqui- | | | | | |
| oxane | | | | | |
| Misc. | add 100 | add 100 | add 100 | add 100 | add 100 |

| | Formula | Formula | Formula | Formula | Formula |
|---|---|---|---|---|---|
| | 11a | 12a | 13a | 14a | 15a |
| Isoparaffin¹⁾ | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polyalkylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 16a | 17a | 18a | 19a | 20a |
| Isoparaffin* | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polyalkylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 21a | 22a | 23a | 24a | 25a |
| Isododecane | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polyalkylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 26a | 27a | 28a | 29a | 30a |
| Isododecane | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polyalkylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 31a | 32a | 33a | 34a | 35a |
| Isoparaffin * | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polyalkylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 36a | 37a | 38a | 39a | 40a |
| Isododecane | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polyalkylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 41a | 42a | 43a | 44a | 45a |
| Isoparaffin¹⁾ | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 46a | 47a | 48a | 49a | 50a |
| Isododecane | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 51a | 52a | 53a | 54a | 55a |
| Isoparaffin¹⁾ | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 56a | 57a | 58a | 59a | 60a |
| Isoparaffin* | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 61a | 62a | 63a | 64a | 65a |
| Isododecane | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c) | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 66a | 67a | 68a | 69a | 70a |
| Isododecane | 60 to 99 | 60 to 98 | 65 to 95 | 65 to 95 | 70 to 90 |
| Ester oil b) | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 71a | 72a | 73a | 74a | 75a |
| Isoparaffin * | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 76a | 77a | 78a | 79a | 80a |
| Isododecane | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Ester oil c)³⁾ | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

| | Formula | Formula | Formula | Formula | Formula |
|---|---|---|---|---|---|
| | 81a | 82a | 83a | 84a | 85a |
| Isododecane | 60 to 99 | 60 to 98 | 65 to 95 | 65 to 95 | 70 to 90 |
| Ester oil b)²⁾ | 0.1 to 20 | 0.5 to 15 | 0.5 to 15 | 1.0 to 10 | 1.0 to 10 |
| Di-n-octyl carbonate | 0.1 to 20 | 0.5 to 15 | 0.5 to 15 | 1.0 to 10 | 1.0 to 10 |
| Trimethylsiloxysilicate | 0.3 to 7.0 | 0.3 to 7.0 | 0.4 to 5.0 | 0.4 to 5.0 | 0.6 to 3.5 |
| Polypropylsilsesquioxane | 0.2 to 3.0 | 0.2 to 3.0 | 0.3 to 2.0 | 0.3 to 2.0 | 0.4 to 1.5 |
| Misc. | add 100 | add 100 | add 100 | add 100 | add 100 |

1) at least one isoparaffin from the group consisting of isodecane, isoundecane, isododecane, isotridecane and isotetradecane
2) tri-fatty acid esters of caprylic acid and capric acid with glycerol
3) esters of carbonic acid with C₆₋₁₂ alkanols

As mentioned above, the cosmetics according to the invention are characterized by above-average care properties on keratin fibers. Another subject matter of this application is therefore the use of a cosmetic according to the invention for keratin fiber care.

The cosmetic according to the invention including the constituents a) to e) is applied to the dry and/or damp keratin fibers. Particular cosmetic advantages are achieved if the cosmetic is applied immediately before, during or after an oxidative or surfactant-based hair treatment.

After being applied to the hair, the cosmetic including the constituents a) to e) preferably remains on the hair until the next time the hair is washed. After the cosmetic according to the invention has been applied, the hair can be dressed and styled by all conventional methods. In particular, the application of heat in the form of a hairdryer, a towel, a drying hood or curling tongs is also possible according to the invention. Of course, further compositions which aid the styling of the hair, for example hairsprays, can also be used.

However, if desired, the composition according to the invention including the constituents a) to e) can also remain on the hair for a few seconds to 30 minutes and then be rinsed off again.

In one variant which is particularly preferred according to the invention, the cosmetic including the constituents a) to e) is prepared as a spray treatment and to this end is combined with a spraying device, preferably a non-aerosol spraying device. When prepared in the administration form that is preferred according to the invention, as a non-aerosol spray application, the spraying pattern and spray mist can be effectively set by way of the viscosity of the cosmetic and/or the choice of spray head and spray pump of the spraying device.

Another subject matter of the present invention is a cosmetic product comprising a cosmetic according to the invention including the constituents a) to e) and also a spraying device, in particular a non-aerosol spraying device for spraying the cosmetic a).

Cosmetics according to the invention including the constituents a) to e) are produced by mixing the ingredients described above.

With regard to further preferred embodiments of the use according to the invention and of the method according to the invention, what has been stated above in respect of the cosmetic according to the invention applies, mutatis mutandis.

Besides the active substances described above, the cosmetics according to the invention including the components a) to c) or a) to e) may include further ingredients. This group of further ingredients includes in particular cosmetically active substances, auxiliaries and additives.

Substances which form oil bodies form a first group of optional ingredients. Examples of these are plant oils, liquid paraffin oils and synthetic hydrocarbons as well as di-n-alkyl ethers having in total between 12 and 36 C atoms and fatty alcohols:
plant oils. Examples of such oils are amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, maize germ oil, almond oil, marula oil, evening primrose oil, olive oil, orange oil, palm oil, peach kernel oil, rape oil, rice oil, sea buckthorn fruit oil, sea buckthorn seed oil, sesame oil, shea butter, soybean oil, sunflower oil, grape kernel oil, walnut oil, wheat germ oil, wild rose oil and the liquid fractions of coconut oil. The proportion by weight of the plant oil based on the total weight of preferred cosmetics is 0.1 to 10% by weight, preferably 0.2 to 8.0% by weight and in particular 0.4 to 5.0% by weight.
liquid paraffin oils and synthetic hydrocarbons as well as di-n-alkyl ethers having in total between 12 to 36 C atoms, in particular 12 to 24 C atoms, such as for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether as well as di-tert-butyl ether, diisopentyl ether, di-3-ethyl decyl ether, tert-butyl n-octyl ether, isopentyl n-octyl ether and 2-methylpentyl n-octyl ether. The compounds 1,3-di-(2-ethylhexyl)cyclohexane (Cetiol® S) and di-n-octyl ether (Cetiol® OE), which are available as commercial products, may be preferred.
fatty alcohols. The group of preferred fatty alcohols includes the C₆ to C₂₂ fatty alcohols, preferably the C₁₀ to C₂₀ fatty alcohols and in particular the C₁₂ to C₁₈ fatty alcohols. The proportion by weight of the fatty alcohol based on the total weight of the preferred cosmetic is 0.1 to 10% by weight, preferably 0.2 to 8.0% by weight and in particular 0.4 to 5.0% by weight.

The total amount of the ingredients of the cosmetics is 100% by weight.

Besides the active substances described above, the cosmetics according to the invention may include further ingredients. This group of further ingredients includes in particular cosmetically active substances, auxiliaries and additives.

As suitable active substances, auxiliaries and additives, mention may be made in particular of additional care substances. As a care substance, the cosmetic may include for example at least one protein hydrolysate and/or a derivative thereof. Protein hydrolysates are product mixtures obtained by acid-, base- or enzyme-catalyzed degradation of proteins (albumins). According to the invention, the term protein hydrolysates is also understood to mean total hydrolysates and also individual amino acids and derivatives thereof and also mixtures of different amino acids. The molecular weight of the protein hydrolysates which can be used according to the invention is between 75, the molecular weight of glycine, and 200,000, and preferably the molecular weight is 75 to 50,000 and very particularly preferably 75 to 20,000 daltons.

As a care substance, the cosmetic according to the invention may also include at least one vitamin, one provitamin, one vitamin precursor and/or one of the derivatives thereof. According to the invention, preference is given to those vitamins, provitamins and vitamin precursors which are usually assigned to groups A, B, C, E, F and H.

Like the addition of glycerol and/or propylene glycol, the addition of panthenol increases the flexibility of the polymer film that is formed when the cosmetic according to the invention is used.

As a care substance, the cosmetics according to the invention may also include at least one plant extract, but also mono- and/or oligosaccharides and/or lipids.

Particularly preferred cosmetics according to the invention are preferably formulated as ready-to-use mixtures including little water. The water content of preferred cosmetics, based on their total weight, is less than 10% by weight, preferably less than 5.0% by weight, particularly preferably less than 1.0% by weight and in particular less than 0.1% by weight, wherein cosmetics which are very particularly preferred include no water.

The compositions, uses and methods according to the invention, and some of the preferred embodiments thereof, are characterized by the following points:
1. A cosmetic including, in a cosmetically acceptable carrier,
   a) at least one isoparaffin from the group consisting of isodecane, isoundecane, isododecane, isotridecane and isotetradecane,
   b) at least one ester oil from the group consisting of tri-fatty acid esters of saturated and/or unsaturated linear and/or branched fatty acids with glycerol,
   c) at least one ester oil c) different from ester oil b).
2. The cosmetic according to point 1, characterized in that the at least one isoparaffin a) is isododecane.
3. The cosmetic according to points 1 or 2, characterized in that the at least one ester oil b) is selected from tri-fatty acid esters of saturated linear and/or branched C₆₋₁₂ carboxylic acids and glycerol.
4. The cosmetic according to point 3, characterized in that the at least one ester oil b) is a mixture of tri-fatty acid esters of caprylic acid and capric acid with glycerol.
5. The cosmetic according to any of points 1 to 4, characterized in that the at least one ester oil c) is selected from symmetrical, unsymmetrical or cyclic esters of carbonic acid with fatty alcohols.
6. The cosmetic according to point 5, characterized in that the at least one ester oil c) is selected from esters of carbonic acid with C₆₋₁₂ alkanols, in particular n-octanol.
7. The cosmetic according to any of points 1 to 6, characterized in that, based on the total weight of components a), b) and c), which add up to 100% by weight, the cosmetic includes
   9.8 to 99.6% by weight component a),
   0.2 to 90% by weight component b),
   0.2 to 90% by weight component c).
8. The cosmetic according to any of points 1 to 7, which is silicone-free.
9. The cosmetic according to any of points 1 to 8, in the form of a hair oil.
10. The cosmetic according to point 1, characterized in that it further includes
   d) at least one trimethylsiloxysilicate and
   e) at least one polyalkylsilsesquioxane.
11. The cosmetic according to point 10, characterized in that the proportion by weight of the isoundecane, isododecane and isotridecane, in particular of the isododecane, based on the total weight of the cosmetic, is 60 to 99% by weight, preferably 65 to 95% by weight and in particular 70 to 90% by weight.
12. The cosmetic according to any of points 10 or 11, characterized in that the at least one isoparaffin a) is isododecane.
13. The cosmetic according to points 10 to 12, characterized in that the at least one ester oil b) is selected from tri-fatty acid esters of saturated linear and/or branched C₆₋₁₂ carboxylic acids and glycerol.
14. The cosmetic according to point 13, characterized in that the at least one ester oil b) is a mixture of tri-fatty acid esters of caprylic acid and capric acid with glycerol.
15. The cosmetic according to point 13, characterized in that the proportion by weight of the ester oil b) based on the total weight of the cosmetic is 0.1 to 20% by weight, preferably 0.5 to 15% by weight and in particular 1.0 to 10% by weight.
16. The cosmetic according to any of points 10 to 15, characterized in that the at least one ester oil c) is selected from symmetrical, unsymmetrical or cyclic esters of carbonic acid with fatty alcohols.
17. The cosmetic according to point 16, characterized in that the at least one ester oil c) is selected from esters of carbonic acid with C₆₋₁₂ alkanols, in particular with n_octanol.
18. The cosmetic according to any of points 10 to 17, characterized in that the proportion by weight of the ester oil c) based on the total weight of the cosmetic is 0.1 to 20% by weight, preferably 0.5 to 15% by weight and in particular 1.0 to 10% by weight.
19. The cosmetic according to any of points 10 to 18, characterized in that the proportion by weight of the trimethylsiloxysilicate d) based on the total weight of the cosmetic is 0.3 to 7.0% by weight, preferably 0.4 to 5.0% by weight and in particular 0.6 to 3.5% by weight.
20. The cosmetic according to any of points 10 to 19, characterized in that the polyalkylsilsesquioxane is a polypropylsilsesquioxane.
21. The cosmetic according to any of points 10 to 20, characterized in that the proportion by weight of the polyalkylsilsesquioxane e) based on the total weight of the cosmetic is 0.2 to 3.0% by weight, preferably 0.3 to 2.0% by weight and in particular 0.4 to 1.5% by weight.
22. The cosmetic according to any of points 10 to 21, characterized in that the weight ratio of components d) and e) is 4:1 to 1:2, preferably 3:1 to 1:1.
23. The cosmetic according to any of points 10 to 22, characterized in that the proportion by weight of components a), b), c), d) and e) is more than 90% by weight, preferably more than 94% by weight and in particular more than 98% by weight of the total weight of the cosmetic.
24. The cosmetic according to any of points 10 to 23, in the form of a spray treatment.
25. A cosmetic product, comprising
   a) a cosmetic according to any of points 10 to 23
   b) a spraying device, in particular a non-aerosol spraying device, for spraying the cosmetic a).
26. The use of a cosmetic according to any of points 1 to 23 for keratin fiber care.
27. A method for producing a cosmetic according to any of points 1 to 23 by mixing the ingredients.
28. A method for treating keratin fibers, characterized in that a cosmetic according to any of points 1 to 23 is applied to dry and/or damp keratin fibers.
29. The method according to point 28, characterized in that the cosmetic is applied immediately before, during or after an oxidative or surfactant-based hair treatment.

### EXAMPLES

All amounts are specified in parts by weight. The following formulations are provided using known methods of preparation.

Hair Oil 1
Isododecane: 88.5
Caprylic acid/capric acid triglyceride: 1.0
Dicaprylyl carbonate: 10.0
Perfume: 0.3
Manila oil: 0.2

Hair Oil 2
Isododecane: 98.5
Caprylic acid/capric acid triglyceride: 5.0
Dicaprylyl carbonate: 5.0
Perfume: 0.3
Lipofructyl Argan LS 9779: 0.2

Spray Treatment 1
Isododecane: 88.7
Caprylic acid/capric acid triglyceride: 5.0
Dicaprylyl carbonate: 5.0
Dow Corning MQ-1640 Flakes 1.0
Perfume: 0.3

The isododecane used may be for example Purolan IDD, which is composed of C₁₂ hydrocarbons in a proportion of at least 98% by weight.

The caprylic acid/capric acid triglyceride used may be a product available under the INCI name Caprylic/Capric Triglyceride.

The dicaprylyl carbonate used may be for example Cetiol CC, which is di-n-octyl carbonate.

The perfume used may be for example Perfume 956046 Vivida Plus II.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic comprising, in a cosmetically acceptable carrier,
a) at least one isoparaffin selected from the group consisting of isodecane, isoundecane, isododecane, isotridecane and isotetradecane,
b) at least one ester oil selected from the group consisting of tri-fatty acid esters of saturated and/or unsaturated linear and/or branched fatty acids with glycerol,
c) at least one ester oil c) different from ester oil b).

2. The cosmetic according to claim 1, wherein the at least one isoparaffin a) is isododecane.

3. The cosmetic according to claim 1, wherein the at least one ester oil b) is selected from tri-fatty acid esters of saturated linear and/or branched C₆₋₁₂ carboxylic acids and glycerol.

4. The cosmetic according to claim 3, wherein the at least one ester oil b) is a mixture of tri-fatty acid esters of caprylic acid and capric acid with glycerol.

5. The cosmetic according to claim 1, wherein the at least one ester oil c) is selected from the group consisting of symmetrical, unsymmetrical or cyclic esters of carbonic acid with fatty alcohols.

6. The cosmetic according to claim 5, wherein the at least one ester oil c) is selected from esters of carbonic acid with C₆₋₁₂ alkanols.

7. The cosmetic according to claim 1, wherein, based on the total weight of components a), b), and c), which add up to 00%, the cosmetic includes component a) comprises 9.8 to 99.6% by weight, component b) comprises 0.2 to 90% by weight, and component c) comprises 0.2 to 90% by weight.

8. The cosmetic according to any of claim 1, wherein the cosmetic is silicone-free.

9. The cosmetic according to any of claim 1, wherein the cosmetic is in the form of a hair oil.

10. The cosmetic according to claim 1, wherein the cosmetic is formulated for care of keratin fibers.
